(19) United States
(12) Patent Application Publication
(10) Pub. No.: US 2018/0003652 A1
(43) Pub. Date: Jan. 4, 2018

(54) METHOD OF THREE-DIMENSIONAL SCANNING USING FLUORESCENCE INDUCED BY ELECTROMAGNETIC RADIATION AND A DEVICE FOR EXECUTING THIS METHOD

(71) Applicant: InsightART s.r.o., Praha 7 (CZ)

(72) Inventors: Josef UHER, Roznov pod Radhostem (CZ); Jan JAKUBEK, Hýskov (CZ)

(73) Assignee: InsightART s.r.o., Praha 7 (CZ)

(21) Appl. No.: 15/544,885

(22) PCT Filed: Jan. 19, 2016
(86) PCT No.: PCT/CZ2016/000009

§ 371 Date: Jul. 20, 2017

(30) Foreign Application Priority Data
Jan. 20, 2015 (CZ) PV 2015-27

(51) Int. Cl. G01N 23/223; G06T 7/62; A61B 5/00; G01N 23/04

(52) U.S. Cl. CPC G01N 23/223; A61B 5/0062; G06T 7/62; G01N 23/043

(57) ABSTRACT

For volumetric analysis of the elemental composition of a measured sample (3) the method of three-dimensional scanning is executing using fluorescence induced by electromagnetic radiation, in which the primary beam (1) of electromagnetic radiation is flattened and is directed at the measured sample (3) in which it irradiates the measured area (6). From the measured area (6) there exits fluorescence radiation, which is almost completely shielded by the shielding means (7) to a secondary beam (9), which is released towards the shielded detector (4) through the permeable area (8) formed in the shielding means (7). The secondary beam (9) projects the image of the measured area (6) onto the shielded detector (4), which records the data of the measured area (6) and subsequently uses the data to obtain an elemental composition of the measured sample (3), including the distribution of concentration of elements in the sample volume.

### FIELD OF THE INVENTION

The invention relates to a method of three-dimensional scanning using fluorescence induced by electromagnetic radiation and a device for executing this method for a volumetric analysis of the elemental composition of the measured samples.

### BACKGROUND OF THE INVENTION

In cases where it was necessary to determine the elemental composition of a measured sample, a non-destructive method of spectrometric analysis was often used. Spectrometric analysis works with physical patterns in which the consequence of the interaction of electromagnetic radiation with the measured sample is studied. Based on the individual unique fluorescence spectra of chemical elements contained in the exiting secondary radiation from the measured sample, it is possible to deduce the elemental composition of the measured sample.

One example is the use of X-ray radiation which, upon impact on the measured sample, causes the fluorescence of atoms in the sample. According to the parameters of the spectrum of the fluorescent radiation, the concentration of chemical elements contained in the measured sample can be determined.

Fluorescence induced by X-ray radiation is used for example in patent U.S. Pat. No. 7,978,820 B2, which describes a device combining X-ray induced X-ray-induced fluorescence and diffraction of the X-ray beam in a crystal lattice of the measured sample. The device includes a source of X-rays from which there exits a polychromatic primary beam of radiation. The beam is directed to the measured sample, where its diffraction occurs and the spectrum of radiation after diffraction is measured. Information is thus obtained on the crystalline structure of the sample. The device is further provided with a detector of secondary fluorescence radiation for a spectrometric analysis of the elemental composition.

The disadvantages of the aforementioned devices consist in that the primary beam irradiates the entire sample, causing fluorescent radiation to exit from the entire volume of the sample. The measurement results therefore contain a concentration of the elements contained in the entire volume of the measured sample. Their placement in the volume of the measured sample cannot be determined.

When examining in particular larger objects, it is often necessary to obtain information on the distribution of concentration of chemical elements on the surface or volume of the sample. This is achieved by irradiating the surface of the measured sample with a narrowly collimated x-ray source point by point. The X-ray spectrometer detects secondary fluorescence radiation at each point. Both the X-ray source and the spectrometer are successively positioned opposite the entire surface of the measured sample, thus obtaining a two dimensional map of the elemental composition of the sample.

Examples of objects where the knowledge of the distribution of elements on the surface or in the volume is required are images. When examining rare works of arts, specifically pigments of applied paints, it is essential that the methods of investigation do not lead to the damage to the work. This is why the X-ray fluorescence analysis is advantageous. Scanning X-ray fluorescence devices for examining paintings are known in which the elemental composition of the painting is analyzed. Such devices have a structure for mounting a planar measured sample. Knowledge of the elemental composition makes the work easier to identify and categorize into a time period, or to be restored.

In cases of paintings painted several times over, it is therefore possible to determine the chemical elements contained in the painting of all layers of the painting simultaneously, but this method is not able to distinguish the various layers of pigments that the painting contains.

The present invention is the creation of a method and device that would be able to analyze the distribution of chemical elements in the volume and which would not lead to damage to the measured sample, which would be suitable for works of art such as paintings and old books, and which would also enable the color reconstruction of multiple-painted paintings according to the positioning of the occurrence of chemical elements used in the creation of the color hues. The invention should also be useful for analyzing integrated circuits and composite materials, for examining the quality of paint layers, analysis of minerals, etc.

### SUMMARY OF THE INVENTION

This objective is solved by a method for three-dimensional scanning using fluorescence induced by electromagnetic radiation and a device for executing this method according to the present invention.

The method of scanning using fluorescence induced by electromagnetic radiation first includes generating a primary beam of electromagnetic radiation from the source. The primary beam is directed onto at least one part of the measured sample, and subsequently by at least one detector, detects the fluorescence electromagnetic radiation exiting from the material of the measured sample. Based on a spectral analysis of the fluorescent radiation, the elemental composition of the measured sample is determined.

The essence of the invention consists in that the shape of the primary beam is flattened so as to have a tabular shape, the flattened primary beam is subsequently pointed towards the measured sample at a defined angle ranging from 0° to 90°. The penetration of the flattened primary beam and the measured sample form a measured area, inside which there emits fluorescence electromagnetic radiation spreading from the measured area to the surrounding environment. The fluorescent electromagnetic radiation is shielded by using a shielding means positioned between the measured sample and the shielded detector. At the same time, the shielding means is provided with at least one permeable area for the centrally symmetrical projection of the secondary beam of fluorescence electromagnetic radiation to a detector. The permeable area creates, on the sensitive area of the shielded detector, an image of the measured part of the sample. Inside the projected image, the impact site of secondary photons on the shielded detector can be uniquely combined with the site of emission of secondary photons from the measured area. The shielded detector measures, in its individual pixels, the intensity and energy of the impacting secondary radiation. Based on the image of the measured shielded detector, on the value of the defined angle of impact of the primary beam, and on the position of the permeable area towards the measured sample and the shielded detector, the composition and distribution of elements are determined in at least part of the volume of the measured sample.

The projection of the measured area using central symmetry on the shielded detector enables scanning using fluorescence radiation within the volume of the measured sample. The resulting calculation provides information about the elemental composition and distribution of elements in the volume of the entire structure of the measured sample, not only data on the existence/nonexistence and the measured concentration of the elements occurring in the measured sample.

In another preferred embodiment of the method of scanning using fluorescence induced by electromagnetic radiation according to the present invention, simultaneously with the scanning of the measured sample the total spectrum of fluorescence electromagnetic radiation is detected by the exposed detector, and the transmission detector also detects the primary beam exiting from the measured sample, in particular its intensity, scattering, and diffraction. To model the volume and map of the distribution of elements in the measured sample, it is appropriate to obtain data on the occurrence and concentration of elements from the exposed detector. It also brings important information about nature of the material about how the primary beam was changed during the course of irradiation of the measured area. By combining data from individual detectors, the structure and composition of the measured sample can be accurately modeled.

In another preferred embodiment of the method of scanning using fluorescence induced by electromagnetic radiation according to the present invention, the measured sample moves during the scanning towards the primary beam to scan the entire volume of the measured sample, or the movement is kinematically reversed. For measurements of samples having a large surface area, e.g. paintings, it is important to divide the sample into multiple measurement areas, whereupon the results of the scanning of the measured areas will complete, in the final modeling, the concentration of elements in the entire volume of the measured sample.

This invention also includes a device for executing the method of scanning using fluorescence induced by electromagnetic radiation.

The device for three-dimensional scanning using fluorescence induced by electromagnetic radiation source includes a primary beam of electromagnetic radiation for irradiating the measured sample and at least one electromagnetic radiation detector for detecting fluorescence electromagnetic radiation exiting from the material of the measured sample.

The essence of the invention consists in that the source of the primary beam is provided with at least one modeling means for flattening the primary beam. Furthermore, the device is provided with an adjustable carrier for the measured object, towards which the primary beam is angularly adjustable to define the angle of impact of the primary beam. The device is also provided with a shielded detector and a shielding means positioned between the measured sample and the shielded detector to prevent the impact of all of the fluorescence radiation onto the shielded detector, wherein the shielding means comprises at least one permeable area for the passage of fluorescence electromagnetic radiation through the shielding means and the projection of the secondary beam onto the shielded detector. The primary beam is modeled into a plate shape and radiates through the measured area of the measured sample. The shielding means allows for the impact of the secondary beam onto the shielded detector in the context of creating an inverted image of the measured area onto the shielded detector by constant central symmetry emerging from the permeable area.

In another preferred embodiment of the device for three-dimensional scanning using fluorescence induced by electromagnetic radiation according to the present invention, the height of the flattened primary beam ranges from 1 μm to 1 mm. The height of the beam determines the size of the measured area, so it is important that it is variable.

In another preferred embodiment of the device for three-dimensional scanning using fluorescence induced by electromagnetic radiation according to the present invention, the source of the primary beam emits at least one type of electromagnetic radiation from the following group: monochromatic X-ray, polychromatic X-ray, gamma radiation. The basic requirement is that the electromagnetic radiation has sufficient energy to initiate fluorescence in the material in the measured sample. The type of radiation is then suitably selected according to the measured sample and the desired results.

In another preferred embodiment of the device for three-dimensional scanning using fluorescence induced by electromagnetic radiation according to the present invention, the modeling means is formed by an X-ray optics and/or collimator. Radiation tends to spread out in all directions from the source that is causing it, so the optics and/or collimator model it into a plate shape of a given height.

In another preferred embodiment of the device for three-dimensional scanning using fluorescence induced by electromagnetic radiation according to the present invention, the shielding means is formed by a material absorbing electromagnetic radiation and the permeable area is formed by an opening, or by X-ray optics, or by a collimator. The shielding means is formed by a material that can absorb electromagnetic radiation and shield the shielded detector, onto which only the secondary beam exiting from the permeable area is projected. The permeable area may be formed only by a hole, but for more intense, higher contrast, and/or a sharper image, it is advisable to use X-ray optics, coded aperture or a collimator.

In another preferred embodiment of the device for three-dimensional scanning using fluorescence induced by electromagnetic radiation according to the present invention, the devices is provided with a transmission detector for detecting changes in the intensity of the primary beam and in its scattering and diffraction, and further is provided with an exposed detector for detecting the total fluorescence radiation. Using data from both detectors, the concentrations of elements in the volume of the measured sample can be more accurately modeled, since a study of the change in the primary beam enables the determination of the physical properties of the material, and a detailed analysis of the concentration of elements from the exposed detector enables a specification of the data on the occurrence of elements in the measured sample volume as determined from the data of the shielded detector.

In another preferred embodiment of the device for three-dimensional scanning using fluorescence induced by electromagnetic radiation according to the present invention, the detector for detecting the electromagnetic radiation is at least one of the following types of detectors: X-ray spectrometer, imaging detector, pixel detector integrating a charge, pixel detector counting individual photons, energy sensitive pixel detector.

In another preferred embodiment of the device for three-dimensional scanning using fluorescence induced by electromagnetic radiation according to the present invention, the adjustable carrier and/or source are motorized to allow continuous measurement of the connected measured areas of the measured sample. For larger measured samples, it is essential to ensure movement so that the individual measured areas tie into each other and are subsequently joined into the final model.

The advantages of the method of three-dimensional scanning using fluorescence induced by electromagnetic radiation, and the device for executing this method, include the possibility of determining the occurrence and concentration of elements in the volume of a measured sample, measuring changes in the parameters of the primary beam that provide information about the material properties of the measured sample, measuring the fluorescence radiation by the exposed detector for a detailed description of the concentration of the elemental composition of the measured sample, and using more types of electromagnetic radiation and more types of detectors.

### DESCRIPTION OF THE DRAWINGS

The invention is more closely illustrated in the following drawings, wherein:

FIG. 1 presents a schematic representation of the device in cross-section,

FIG. 2 shows an axonometric schematic drawing of the scanning of the measured object,

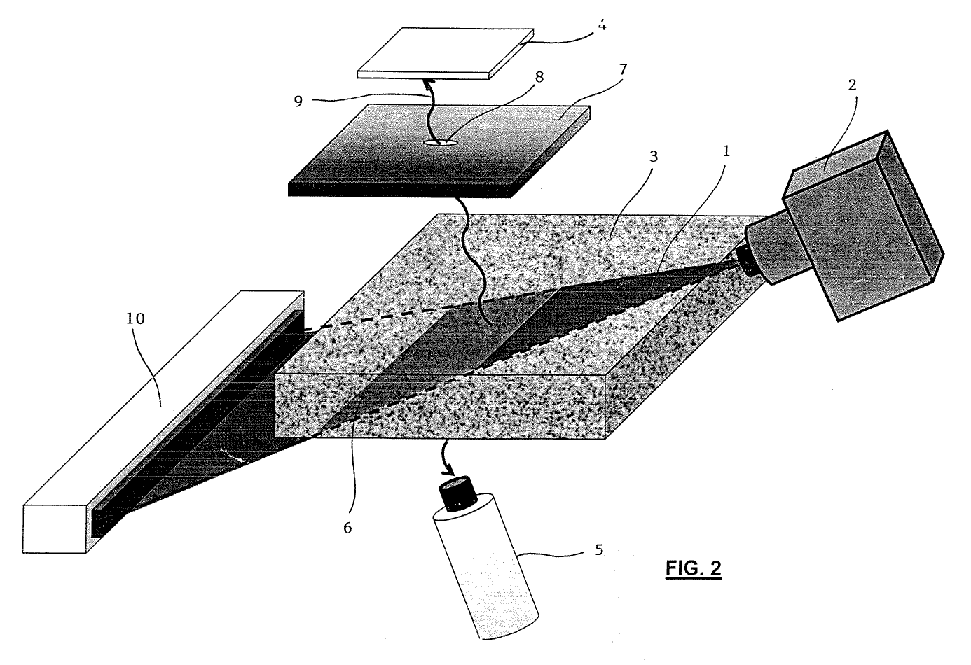

### EXAMPLES OF THE PREFERRED EMBODIMENTS OF THE INVENTION

It is understood that the hereinafter described and illustrated specific examples of the realization of the invention are presented for illustrative purposes and not as a limitation of the examples of the realization of the invention to the cases shown herein. Experts who are familiar with the state of technology shall find, or using routine experimentation will be able to determine, a greater or lesser number of equivalents to the specific realizations of the invention, which are specifically described here. These equivalents shall also be included into the scope of the patent claims.

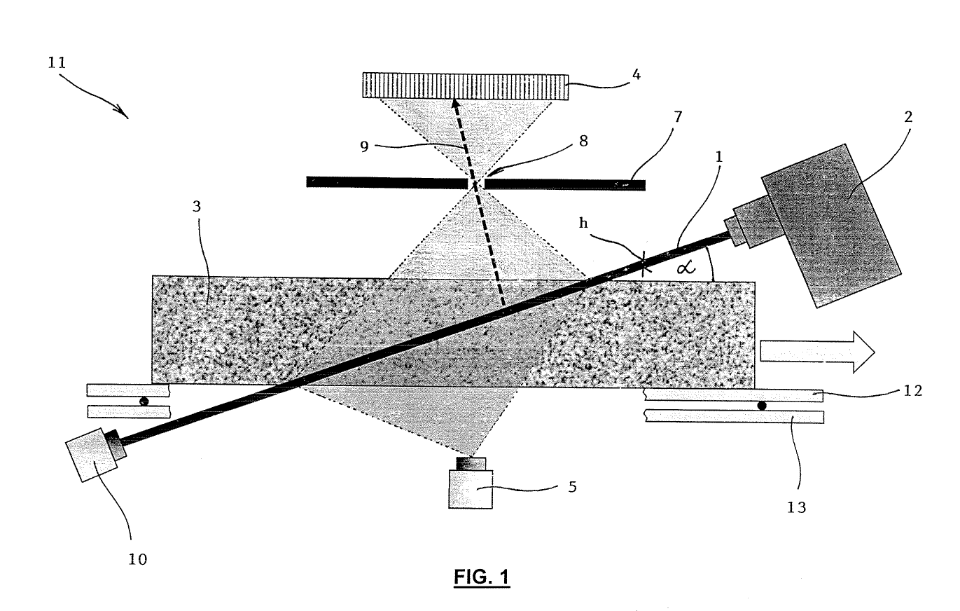

FIG. 1 is a schematic illustration of the device 11 for three-dimensional scanning using fluorescence induced by electromagnetic radiation. The basis of the device 11 is a metal frame 13 to which the various components of the device 11 are fixed. The source 2 of the primary beam 1 is formed, in this particular example, by an X-ray tube, before which there is positioned collimator and X-ray optics. From the source 2 there radiates the primary beam 1 which is straight, flattened, its height h is 15 μm, and its width is modeled in the range of millimeters or centimeters as appropriate for the measurement. This is achieved, for example, by collimation, X-ray optics, or another method (e.g. by a synchrotron). The primary beam 1 fails on the measured sample 3.

The frame 13 and adjustable carrier 12 allow for the precise positioning of the measured sample 3 towards the source 2 and towards the primary beam 1 using motors, so the measured sample 3 can be irradiated successively in sections. In other embodiments of the invention, devices with an arbitrary principle of generating electromagnetic radiation (e.g. X-ray tube, synchrotron, radionuclide source, etc.) may serve as the source 2 of the primary beam 1. The basic condition is that the energy of the primary beam 1 is sufficient to induce fluorescence in the measured sample 3.

The measured sample 3 is mounted on the adjustable carrier 12. The carrier 12 is a table on which the measured sample 3 is laid or fixed, and secured against arbitrary movement. The carrier 12 is adjustable to correct inaccuracies when placing the measured sample 3 into the device 11.

In the path of the primary beam 1 there lies a transmission detector 10, which detects the exiting primary beam 1 of the measured sample 3. The detector 10 monitors the intensity of the primary beam 1, its dispersion and bending, thereby obtaining data on the nature of the material of the measured sample 3.

Upon penetration of the primary beam 1 through the measured sample 3, the irradiated area 6 is measured and emits fluorescence radiation, which spreads in all directions. Inside the device 11, there is therefore also stored an exposed detector 5 which detects this radiation and sends the data to be processed for each measured area 6 of the sample 3.

Part of the fluorescence radiation from the measurement area 6 spreads towards the shielded detector 4 which is hidden behind the shielding means 7. The shielding means 7 absorbs the fluorescence radiation along its entire area except for the permeable area 8 which allows for the penetration of the photons of the fluorescence radiation forming a secondary beam 9 continuing to the shielded detector 4. A pinhole camera is thus created for X-rays. A knowledge of the direction of the primary beam 1 during the irradiation of the measured sample 3 allows, from geometric dependencies, for the determination of the site in the material of the measured area 6 of the measured sample area 3 from where the fluorescence radiation was emitted. The shielding means 7 is formed by a shielding metal (e.g. lead or tungsten) and the permeable area 10 is a normal hole of small dimensions, or in another different example of an embodiment is formed by an X-ray optics, coded aperture or a collimator.

The primary beam 1 impacting below the angle α of size 10° passes through the measured sample 3 and exits from the measured sample 3. It then impacts upon the detector 10, which measures how the primary beam 1 was affected by its passage through the measured sample 3. Simultaneously with the passage of the primary beam 1 through the material of the measured sample 3 there occurs emission of fluorescence radiation. The radiation spreads in all directions, including the direction towards the shielded detector 4 stored behind the shielding means 7. Through the permeable area 8 there penetrates part of the fluorescence radiation forming a secondary beam 9 to the detection surface of the position-sensitive shielded detector 4. Given the knowledge of the orientation of the primary beam 1 towards the measured object 3, it is possible to read, from the detector 4, the data for the entire course of the primary beam 1 through the material of the measured sample 3 along its height and width. By moving the sample 3 in relation to the detector 4 and to the primary beam 1, information is then obtained from the entire volume of the measured sample 3.

Detectors 4 and 10 include either a single position- and energy-sensitive X-ray imaging detector, or several detection chips arranged in a common field. The detection chips are, for example, Timepix detectors enabling the measurement of the position and energy of the impacting radiation.

Detector 10 measures the attenuation of the primary beam 1 after its passage through the measured sample 3. It thus creates an X-ray image of the measured sample 3 during the scanning transmission. Detector 10 may be position-sensitive, and/or spectrometric same as detector 4. It then provides further information about the composition of the measured sample 3. Detector 10 can also be purely spectrometric, like detector 5. If it is a position-sensitive, it can also provide information about the photons of the primary beam 1 scattered through the sample outside this beam 1.

Detector 5 measures the total fluorescence spectrum emitted from the entire irradiated volume of the sample 3. This detector 5 is not position-sensitive, but has a good energy resolution. An analysis of the spectrum measured by the detector 5 provides an overall concentration of elements in the irradiated volume (i.e. without information on distribution in space). The detector 5 may be, for example, an SDD (silicon-drift detector) type.

Information from detectors 5 and 10 may be used separately (transmission image and total elemental composition). Or it may be used in the analysis of the spectra measured in the pixels of detector 4. An overall knowledge of the elemental composition obtained by detector 5 will reduce the number of free parameters in the analysis of data from detector 4. Data from detector 10 can be used to obtain a correction for self-shielding in the sample 3 when determining the concentrations of elements from the spectra in detectors 4 and 5.

Detectors 4, 5, 10 are adjustable on the frame 2, either positionable by handles or by motors.

During the scanning, the measured sample 3 can be moved on the carrier 12, or the detectors 4, 5, 10 and the source 2 may be moved in individual steps. The decisive factor is the size and shape of the measured sample 3.

### INDUSTRIAL APPLICABILITY

The method and device for three-dimensional scanning according to the invention shall find application in the field of restoration of works of art, in the field of printed circuit boards, integrated circuits, non-destructive testing, or in the field of analysis of layered composite materials.

### OVERVIEW OF THE POSITIONS USED IN THE DRAWINGS

1 primary beam of electromagnetic radiation
2 source of the primary beam of electromagnetic radiation
3 measured sample
4 shielded detector
5 exposed detector
6 measured area
7 shielding means
8 permeable area
9 secondary beam of fluorescence electromagnetic radiation
10 transmission detector
11 device for three-dimensional scanning
12 adjustable holder for the measured sample
13 frame for attaching the parts of the device
α angle between the primary beam and the measured sample
h height of the flattened primary beam

1. A method of scanning using fluorescence induced by electromagnetic radiation in which there is generated a primary beam (1) of electromagnetic radiation from a source (2), the primary beam (1) is directed to at least one part of the measured sample (3), and by using at least one detector (4, 5) fluorescence electromagnetic radiation is detected exiting from the material of the measured sample (3), and on the basis of its spectral analysis the elemental composition of the measured sample (3) is determined, characterized in that the shape of the primary beam (1) is flattened, the flattened primary beam (1) is directed to the measured sample (3) at an angle (α) whose magnitude ranges from 0° to 90°, whereupon the penetration of the flattened primary beam (1) and the measured sample (3) forms the measured area (6), inside which there is emitted fluorescent electromagnetic radiation, the fluorescence electromagnetic radiation is shielded using a shielding means (7) positioned between the measured sample (3) and the shielded detector (4), wherein the shielding means (7) is provided with at least one permeable area (8) to create a secondary beam (9) of fluorescence electromagnetic radiation and for a clear connection of the site of radiation of the secondary beam (9) of the measured area (6) and the site of impact of the secondary beam (9) on the shielded detector (4), subsequently on the shielded detector (4) there is detected a secondary beam (9) exiting from the permeable area (8), whereupon on the basis of the shielded detector (4) of the measured data, on the value of the angle (α) and the position of the permeable region (8) towards the measured sample (3) and/or the shielded detector (4), the elemental composition is modeled in at least part of the volume of the measured sample (3).

2. A method of scanning according to claim 1, characterized in that simultaneously with the scanning of the measured sample (3) the overall spectrum of the fluorescence electromagnetic radiation is detected by the exposed detector (5), and simultaneously the transmission detector (10) detects the primary beam (1) exiting from the measured sample (3), in particular its intensity, scattering, and diffraction.

3. A method of three-dimensional scanning according to claim 1 or 2, characterized in that the measured sample (3) is moved during scanning towards the primary beam (1) so that the entire volume of the measured sample (3) may be scanned, or that the kinematic motion is reversed.

4. A device (11) for three-dimensional scanning using fluorescence induced by electromagnetic radiation according to the method stated in at least one of claims 1 to 3, comprising a source (2) of the primary beam (1) of electromagnetic radiation and at least one detector (4, 5, 10) of the electromagnetic radiation, characterized in that the source (2) of the primary beam (1) is provided with at least one modeling means for flattening the primary beam (1), the device (11) is provided with an adjustable carrier (12) for the measured object (3), towards which the primary beam (1) is angularly adjustable, further the device (11) is provided with a shielding means (7) positioned between the measured sample (3) and the shielded detector (4), wherein the shielding means (7) has at least one permeable area (8) for the passage of fluorescence electromagnetic radiation through the shielding means (7) and for the generation of a secondary beam (9).

5. A device according to claim 4, characterized in that the height (h) of the flattened primary beam (1) is in the range from 1 μm to 1 mm.

6. A device according to claim 4 or 5, characterized in that the source (2) of the primary beam (1) emits at least one type of electromagnetic radiation from the following group: monochromatic X-ray, polychromatic X-ray, gamma radiation.

7. A device according to any of claims 4 to 6, characterized in that the modeling means is formed by X-ray optics and/or a collimator.

8. A device according to any of claims 4 to 7, characterized in that the shielding means (7) is formed by a material absorbing electromagnetic radiation, and the permeable area (8) is formed by an opening, or X-ray optics, or a collimator.

9. A device according to any of claims 4 to 8, characterized in that it is provided with a transmission detector (10) for detecting changes in the intensity of the primary beam (1), and its scattering and diffraction, and further is provided with an exposed detector (5) for detecting total fluorescence radiation.

10. A device according to any of claims 4 to 9, characterized in that the detector (4, 5, 10) for detecting electromagnetic radiation is at least one of the following types of detector: X-ray spectrometer, imaging detector, pixel detector integrating a charge, pixel detector counting individual photons, energy-sensitive pixel detector.

11. A device according to any of claims 4 to 10, characterized in that the adjustable carrier (12) and/or source (2) is motorized to allow for continuous measurement of the connected measured areas zone (6) of the measured sample (3).
